(19) United States
(12) Patent Application Publication
(10) Pub. No.: US 2026/0114728 A1
(43) Pub. Date: Apr. 30, 2026

(54) CORNEAL ANALYSIS SYSTEMS AND METHODS EMPLOYING BRILLOUIN SPECTROSCOPY FOR DETECTION OF SUBCLINICAL KERATOCONUS

(71) Applicants: University of Maryland, College Park, College Park, MD (US); The Cleveland Clinic Foundation, Cleveland, OH (US)

(72) Inventors: James Bradley RANDLEMAN, Westlake, OH (US); Giuliano SCARCELLI, Washington, DC (US)

(21) Appl. No.: 19/368,172

(22) Filed: Oct. 24, 2025

Related U.S. Application Data
(60) Provisional application No. 63/711,240, filed on Oct. 24, 2024.

(51) Int. Cl. A61B 3/107; A61B 3/00; A61B 3/10; A61B 5/00

(52) U.S. Cl. CPC A61B 3/107; A61B 3/0008; A61B 3/1005; A61B 5/0066; A61B 5/0075

(57) ABSTRACT

Corneal thickness and/or curvature of a patient's eye can be measured. Based on the measured corneal geometry characteristic, a focal test region for the eye can be defined. Brillouin spectroscopy measurements can be obtained of the eye for different measurement points. A Brillouin focality index (BFI) can be calculated based at least in part on one or more of the Brillouin spectroscopy measurements obtained for measurement points in the focal test region. The eye can be classified as exhibiting subclinical keratoconus based at least in part on the calculated BFI.

### CROSS-REFERENCE TO RELATED APPLICATIONS

The present application claims the benefit of and priority under 35 U.S.C. § 119 (e) to and is a non-provisional of U.S. Provisional Application No. 63/711,240, filed Oct. 24, 2024, entitled “Corneal Planning System,” which is hereby incorporated by reference herein in its entirety.

### STATEMENT REGARDING FEDERALLY SPONSORED RESEARCH

This invention was made with government support under EY028666 awarded by the National Institutes of Health. The government has certain rights in the invention.

### FIELD

The present disclosure relates generally to biomechanical imaging of eyes, and more particularly, to analysis of the cornea of an eye using Brillouin spectroscopy, for example, for detecting subclinical keratoconus.

### BACKGROUND

Deterioration of corneal biomechanics is the root cause of corneal ectatic disorders, such as keratoconus (KC) and postoperative ectasia after laser vision correction procedures (e.g., laser in situ keratomileusis (LASIK)). In these conditions, the mechanical balance between intraocular forces and corneal resistance is disrupted, resulting in corneal thinning and warpage that causes dramatic alterations in corneal refractive power. While clinically manifest keratoconus has highly recognizable morphological features identifiable using a variety of available technologies, differentiation of subclinical keratoconus from normal corneas continues to be challenging. Roberts and Dupps proposed that focal corneal weakening, rather than generalized corneal weakening initiates a cycle of corneal decompensation (the Corneal Biomechanical Decompensation (CBD) Cycle), in which a focally reduced corneal elastic modulus initiates a cycle of increased focal strain and stress redistribution, with subsequent focal thinning and asymmetric anterior curvature that escalate with disease progression. Recently, Roberts and colleagues demonstrated that focal maximal strain colocalizes with thinnest corneal point minimal in normal corneas, while the predominant biomechanical driver evolves to minimum stress at the point of greatest curvature in keratoconus. An updated theory for the CBD cycle thus suggests that focal biomechanical weakening at the thinnest corneal point may precede asymmetric anterior curvature changes.

However, it has not yet been possible to obtain data confirming the updated CBD cycle, since conventional devices are unable to provide focal biomechanical measurements. For example, current corneal imaging devices (e.g., Placido, Scheimpflug, optical coherence tomography (OCT)) provide morphologic data only. Moreover, available biomechanical measurement devices (e.g., Ocular Response Analyzer (ORA), Corneal Visualization Scheimpflug Technology (Corvis-ST)) can only provide generalized corneal data and are thus insensitive to localized altered modulus. While multiple screening devices, metrics, and strategies have been developed for subclinical keratoconus detection, these continue to demonstrate limited efficacy in detecting subclinical keratoconus.

Embodiments of the disclosed subject matter may address one or more of the above-noted problems and disadvantages, among other things.

### SUMMARY

Embodiments of the disclosed subject matter provide systems and methods employing Brillouin spectroscopy to analyze the cornea of a patient's eye, in particular, to measure focal corneal biomechanical data in vivo and provide early detection of keratoconus (i.e., subclinical keratoconus (SKC)), which may otherwise be difficult or impossible to detect with existing imaging or biomechanical measurement instruments. In some embodiments, Brillouin spectroscopy measurements are obtained for multiple points of a patient's eye, and each measurement can yield a Brillouin frequency shift, or other Brillouin reading, that provides a quantitative estimate of the longitudinal modulus at the respective measurement point. Measurements of corneal geometry can also be obtained, and a point in the cornea with a measured extrema (e.g., thinnest corneal point (TCP) and/or maximum corneal curvature (MCC)) can be identified. In some embodiments, a focal test region can be defined based on the identified extrema point, and Brillouin spectroscopy measurements from points only within the defined focal test region can be used to calculate a Brillouin focality index (BFI). Based on the BFI, the eye can be classified as exhibiting SKC.

In some embodiments, the focal test region can be defined prior to obtaining the Brillouin spectroscopy measurements, and the Brillouin spectroscopy measurements may be obtained for points only within the focal test region. In some embodiments, this smaller region for acquiring Brillouin measurements can be particularly beneficial within the practical operations of an ophthalmology clinic, because it can significantly shorten the total time required to perform Brillouin analysis of a given eye (e.g., <5 minutes, for example, ≤3 minutes, or even≤1 minute). Indeed, Brillouin imaging sessions with conventional Brillouin systems are currently lengthy (e.g., >10-20 minutes) and thus are difficult to integrate within the clinical pipeline and/or induce patient dissatisfaction.

In one or more embodiments, a system can comprise a first imaging device, a Brillouin spectroscopy device, and a controller. The first imaging device can be configured to optically interrogate an eye of a patient. The controller can be operatively coupled to the first imaging device and the Brillouin spectroscopy device so as to communicate therewith and control operational aspects thereof. The controller can comprise one or more processors and one or more computer readable storage media storing computer readable instructions that, when executed by the one or more processors, cause the system to: (a) measure one or more corneal geometry characteristics of the eye via the first imaging device; (b) define a focal test region for the eye based at least in part on the measured one or more corneal geometry characteristics; (c) obtain a plurality of Brillouin spectroscopy measurements of the eye for different measurement points via the Brillouin spectroscopy device; (d) calculate a Brillouin focality index (BFI) based at least in part on one or more of the plurality of Brillouin spectroscopy measurements obtained for measurement points in the focal test region; and (e) classify the eye as exhibiting subclinical keratoconus based at least in part on the calculated BFI.

In one or more embodiments, a method can comprise optically interrogating an eye of a patient so as to measure one or more corneal geometry characteristics. The method can further comprise defining a focal test region for the eye based at least in part on the measured one or more corneal geometry characteristics. The method can also comprise obtaining a plurality of Brillouin spectroscopy measurements of the eye for different measurement points. The method can further comprise calculating a Brillouin focality index (BFI) based at least in part on one or more of the plurality of Brillouin spectroscopy measurements obtained for measurement points in the focal test region. The method can also comprise classifying the eye as exhibiting subclinical keratoconus based at least in part on the calculated BFI.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will hereinafter be described with reference to the accompanying drawings, which have not necessarily been drawn to scale. Where applicable, some elements may be simplified or otherwise not illustrated in order to assist in the illustration and description of underlying features. For example, in some figures, the propagation of light has not been shown or has been illustrated using block arrows or solid/dashed lines rather than employing ray diagrams. Throughout the figures, like reference numerals denote like elements.

FIG. 1A is a simplified schematic diagram of a configuration for a corneal analysis system, according to one or more embodiments of the disclosed subject matter.

FIG. 1B illustrates detection aspects in front and cross-sectional views of a patient's eye for determining a Brillouin Focality Index (BFI), according to one or more embodiments of the disclosed subject matter.

FIG. 1C is a simplified schematic diagram of another configuration for a corneal analysis system, according to one or more embodiments of the disclosed subject matter.

FIG. 1D illustrates an exemplary diagnostic device based on the configuration of FIG. 1C.

FIG. 2A illustrates aspects of a Brillouin spectroscopy modality, according to one or more embodiments of the disclosed subject matter.

FIG. 2B illustrates an exemplary configuration of an interrogating light source for use in a Brillouin spectroscopy modality, according to one or more embodiments of the disclosed subject matter.

FIG. 2C illustrates an arrangement of optical components for a detection arm of a Brillouin spectroscopy modality, according to one or more embodiments of the disclosed subject matter.

FIG. 3 is a simplified schematic diagram of a corneal analysis system for detecting subclinical keratoconus, according to one or more embodiments of the disclosed subject matter.

FIG. 4A is a process flow diagram of a method for detection of subclinical keratoconus (SKC) via Brillouin spectroscopy measurements, according to one or more embodiments of the disclosed subject matter.

FIG. 4B is a process flow diagram of a method for detection of subclinical keratoconus (SKC) via Brillouin spectroscopy measurements, according to one or more embodiments of the disclosed subject matter.

FIG. 4C shows aspects of Brillouin spectroscopy measurements at multiple corneal depths, according to one or more embodiments of the disclosed subject matter.

FIG. 5 depicts a generalized example of a computing environment in which the disclosed technologies may be implemented.

FIGS. 6A-6B show measured Brillouin maps for normal and eyes exhibiting SKC, respectively.

FIG. 7 shows pachymetry and Brillouin measurement maps for an eye exhibiting subclinical keratoconus.

FIGS. 8A-8C show lateral positions for measured thinnest and softest points for normal eyes, eyes suspected of SKC, and eyes exhibiting KC, respectively.

FIG. 8D shows lateral positions for measured softest points when the map is centered on the corresponding thinnest measurement point for normal eyes and eyes suspected of SKC.

FIGS. 9A-9D shows results of different measurement and determination stages in calculating BFI for an eye.

### DETAILED DESCRIPTION

### General Considerations

For purposes of this description, certain aspects, advantages, and novel features of the disclosed subject matter are described herein. The disclosed methods and systems should not be construed as being limiting in any way. Instead, the present disclosure is directed toward all novel and nonobvious features and aspects disclosed herein, alone and in various combinations and sub-combinations with one another. The methods and systems are not limited to any specific aspect or feature or combination thereof, nor do the disclosed aspects require that any one or more specific advantages be present, or problems be solved. The technologies from any aspect or example can be combined with the technologies described in any one or more of the other aspects or examples. In view of the many possible aspects to which the principles of the disclosed technology may be applied, it should be recognized that the illustrated aspects of the disclosure are exemplary only and should not be taken as limiting the scope of the disclosed technology.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one skilled in the art.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person skilled in the art to have a more definitive construction, the numerical parameters set forth are approximations that may depend on the desired properties sought and/or limits of detection under standard test conditions/methods, as known to those skilled in the art. When directly and explicitly distinguishing aspects from discussed prior art, the numbers are not approximates unless the word “about.” “substantially.” or “approximately” is recited. Whenever “substantially.” “approximately.” “about.” or similar language is explicitly used in combination with a specific value, variations up to and including 10% of that value are intended, unless explicitly stated otherwise.

Directions and other relative references may be used to facilitate discussion of the drawings and principles herein but are not intended to be limiting. For example, certain terms may be used such as “inner.” “outer.” “upper,” “lower,” “top.” “bottom,” “interior,” “exterior,” “left.” right.” “front.” “back,” “rear,” and the like. Such terms are used, where applicable, to provide some clarity of description when dealing with relative relationships, particularly with respect to the illustrated aspects. Such terms are not, however, intended to imply absolute relationships, positions, and/or orientations. For example, with respect to an object, an “upper” part can become a “lower” part simply by turning the object over. Nevertheless, it is still the same part, and the object remains the same.

As used herein, “comprising” means “including,” and the singular forms “a” or “an” or “the” include plural references unless the context clearly dictates otherwise. The term “or” refers to a single element of stated alternative elements or a combination of two or more elements unless the context clearly indicates otherwise.

Although there are alternatives for various components, parameters, operating conditions, etc. set forth herein, that does not mean that those alternatives are necessarily equivalent and/or perform equally well. Nor does it mean that the alternatives are listed in a preferred order, unless stated otherwise. Unless stated otherwise, any of the groups defined below can be substituted or unsubstituted.

Unless explained otherwise, all technical and scientific terms used herein have the same meaning as commonly understood to one skilled in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Features of the presently disclosed subject matter will be apparent from the following detailed description and the appended claims.

### Introduction

Disclosed herein are systems and methods for evaluating biomechanical properties of an eye of a patient (e.g., a human or animal) via Brillouin spectroscopy measurements, in particular, to provide detection (e.g., early detection) of a progressive eye disease, for example, subclinical keratoconus (SKC). Three-dimensionally localized focally reduced corneal modulus can provide an early indication of SKC. This focal region co-localizes laterally (in two-dimensions) with an extrema of the corneal morphology (also referred to herein as corneal geometry) and within the anterior corneal stroma (in a depth direction). In some embodiments, the focally reduced corneal modulus can be detected via Brillouin spectroscopy measurements, and regional pachymetry and/or corneal curvature data can be obtained in addition to the focal mechanical modulus provided by the Brillouin spectroscopy modality, for example, to provide a comprehensive and sufficient dataset for both diagnostic evaluation and the planning of treatments (e.g., refractive surgery, diagnostic evaluation, laser vision correction, corneal cross-linking treatment for refractive error and keratoconus (KC), and/or other corrective treatments).

In some embodiments, the Brillouin spectroscopy measurements of a patient's eye are indicative of a longitudinal modulus at the measurement point, which, while different from the Young's modulus of the interrogated material, can be interpreted as an indication of a decrease (or increase) in biomechanical stiffness of the measured point in the cornea. In some embodiments, Brillouin spectroscopy measurements can be obtained for a region (e.g., a focal test region) of the cornea most likely to exhibit a focally reduced corneal modulus and can be used to calculate a Brillouin Focality Index (BFI), which can be used to determine that an eye exhibits SKC.

In some embodiments, a focal test region can be defined based on measurement of an extrema of the corneal geometry, for example, a region surrounding a lateral point in the cornea having a minimum thickness (e.g., a thinnest corneal point (TCP)) or a lateral point in the cornea having a maximum curvature (e.g., a maximum curvature point (MCP)). In some embodiments, the Brillouin spectrometry measurements can be obtained only for measurement points within this focal test region. By limiting the region for Brillouin spectroscopy measurements, the number of measurement points can be increased and/or the time required to obtain and process sufficient measurement points to calculate the BFI can be reduced. Alternatively, in some embodiments, the Brillouin spectrometry measurements can be obtained for measurement points across the eye, including points outside the focal test region, but the processing to calculate the BFI may only take into account measurements from points within the focal test region. While the time to acquire the Brillouin spectroscopy measurements may be increased in such embodiments, time savings may nevertheless be realized by virtue of processing a reduced number of Brillouin measurement points to calculate the BFI.

### Corneal Analysis Systems

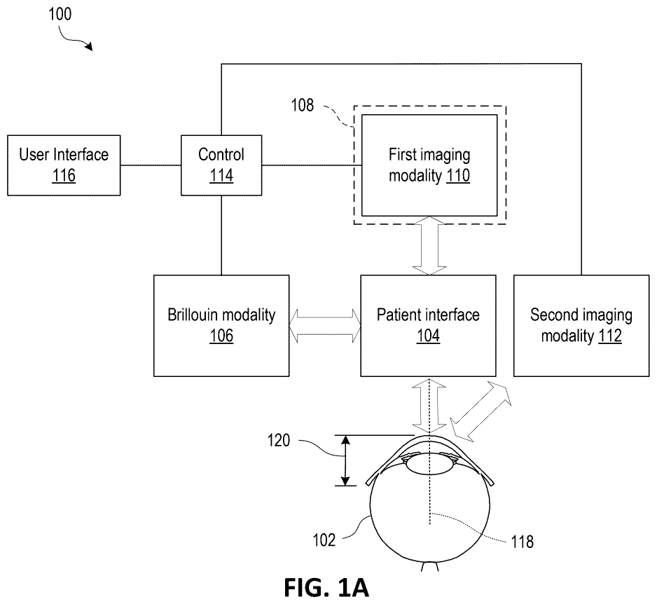

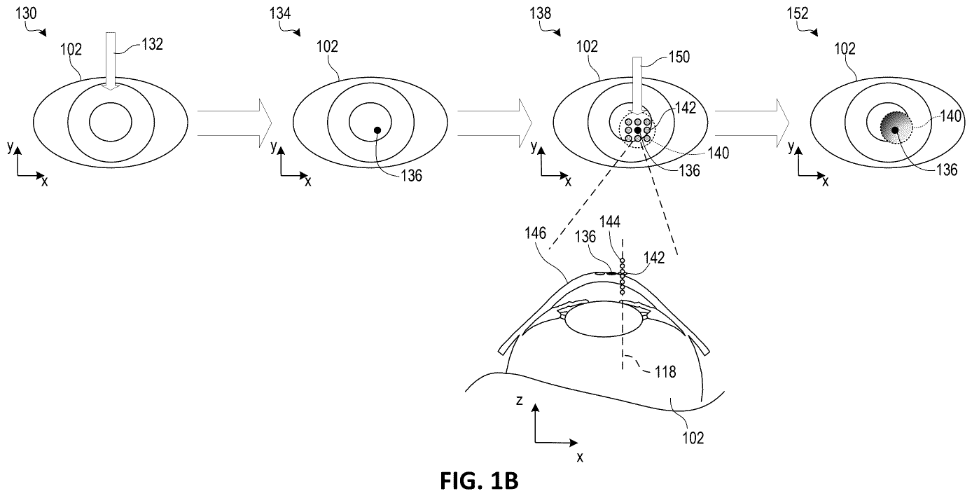

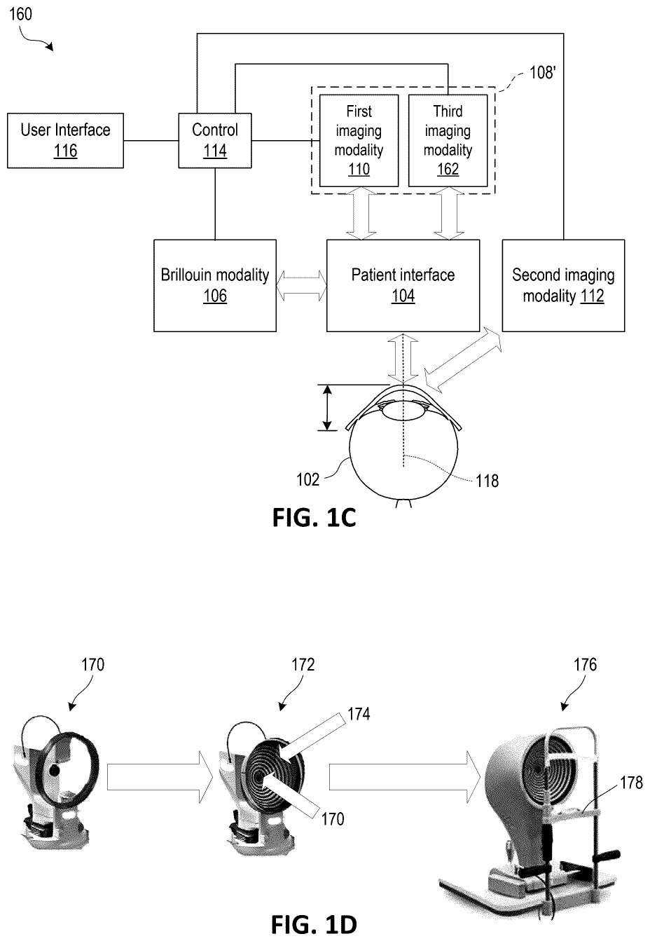

Referring to FIG. 1A, a corneal analysis system 100 for detecting SKC in an eye 102 can include a patient interface 104, a Brillouin spectroscopy measurement modality 106, and a corneal geometry measurement module 108. In some embodiments, the corneal geometry module 108 can include a first imaging modality 110 configured to obtain a measurement of a first corneal geometry of the eye 102. For example, the first imaging modality 110 can provide pachymetry via a non-contact optical method, such as optical coherence tomography (OCT), Scheimpflug imaging, or slit-scanning technology. In some embodiments, the first imaging modality 110 is a full-view OCT, which can provide a 3-D reconstruction of the corneal structure and thus can enable the acquisition of regional pachymetry measurements and Brillouin shift mechanical imaging in one device. Alternatively, in some embodiments, the first imaging modality 110 can provide keratometry using a non-contact optical method, such as Placido ring imaging or other corneal topographic device. In some embodiments, the first imaging modality 110 can also be configured to measure depth 120 (e.g., along axial direction 118) of a measurement point (e.g., focal point) in the eye for a corresponding Brillouin spectroscopy measurement by modality 106. The corneal analysis system 100 can also include a second imaging modality 112 configured to measure lateral locations (e.g., in a plane perpendicular to axial direction 118, for example, a plane tangential to a curvature of the front of the cornea) and/or track movement of the eye 102 during measurements. In some embodiments, the second imaging modality 112 comprises an optical imaging system, such as but not limited to a single lens imaging system.

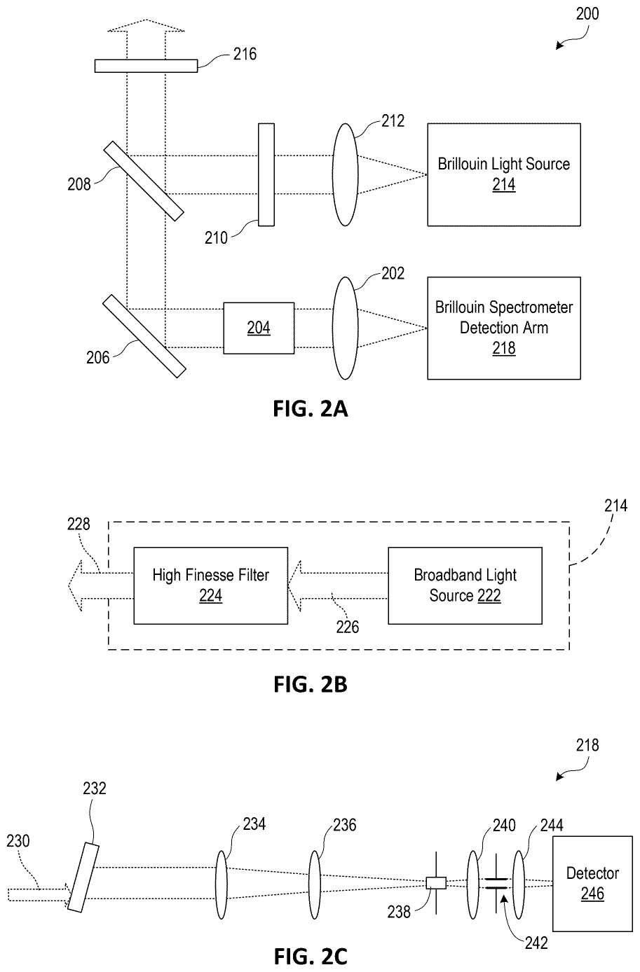

In some embodiments, the Brillouin spectroscopy measurement modality 106 can have a configuration similar to setup 200 of FIG. 2A, a light source similar to source 214 of FIG. 2B, and/or a detection arm similar to detection arm 218 of FIG. 2C, as described in further detail below. Alternatively, in some embodiments, the Brillouin spectroscopy measurement modality 106 can have a configuration (or aspects thereof) as described in International Publication No. WO 2024/064142, published Mar. 28, 2024, and entitled “Brillouin Spectroscopy Systems and Methods for Detection of Subclinical Keratoconus.” Alternatively or additionally, in some embodiments, the Brillouin spectroscopy measurement modality 106 can have a configuration (or aspects thereof) as described in any of: “Noncontact three-dimensional mapping of intracellular hydro-mechanical properties by Brillouin microscopy.” Nature Methods, December 2015, 12 (12): pp. 1132-34; U.S. Pat. No. 12,019,018, issued Jun. 25, 2024, and entitled “Full-field Brillouin microscopy systems and methods,” U.S. Pat. No. 11,408,770, issued Aug. 9, 2022, and entitled “Brillouin imaging devices, and systems and methods employing such devices;” U.S. Pat. No. 11,143,555, issued Oct. 12, 2021, and entitled “Methods and devices for reducing spectral noise and spectrometry systems employing such devices:” U.S. Pat. No. 11,060,912, issued Jul. 13, 2021, and entitled “Multi-stage parallel spectroscopy systems and methods;” U.S. Pat. No. 10,989,591, issued Apr. 27, 2021, and entitled “Methods and arrangements to enhance optical signals within aberrated or scattering samples:” and U.S. Publication No. 2025/0044152, published Feb. 6, 2025, and entitled “Multiplexed Brillouin microscopy systems and methods,” which spectrometer configurations are incorporated herein by reference.

In some embodiments, the patient interface 104 can be constructed to interact with the patient, or the eye thereof, for example, by providing one or more features that align and/or maintain a position of the eye with respect to a focal point of the system 100. For example, the patient interface 104 can include a chin rest (e.g., that accepts a chin of a human patient during interrogation), a bite bar (e.g., that the human patient can bite during interrogation), a forehead rest (e.g., that abuts the forehead of the human patient during interrogation), or any other feature that aligns or positions the head of the patient or a portion thereof (e.g., the eye 102). In some embodiments, the patient interface 104 can include one or more optical components (e.g., polarizers, beam splitters, lenses, mirrors, couplers, filters, etc.) that route and/or scan light from the different modalities 106, 110 to the eye 102 and light from the eye 102 to the different modalities 106, 110. In the illustrated example, light interactions between the second imaging modality 112 and the eye 102 do not pass through the patient interface 104. However, it is possible for the second imaging modality 112 to interact with the eye 102 via the patient interface 104, according to one or more contemplated embodiments.

In some embodiments, the corneal analysis system 100 can also include a controller 114 operatively coupled to and configured to control operation of components of the system 100, for example, the Brillouin spectroscopy measurement modality 106, the first imaging modality 110, and the second imaging modality 112. In some embodiments, the controller 114 can synchronize operation of the different modalities 106, 110, 112, and/or can combine together data from the separate modalities. Alternatively or additionally, in some embodiments, controller 114 can control operation of the modalities 106, 110, 112 to acquire data, and a separate system (not shown) can process the data (e.g., to process Brillouin spectroscopy measurements from a focal test region, calculate a Brillouin focality index, and/or diagnose or otherwise characterize the eye, etc.) receive from the controller 114 (e.g., independent of the data acquisition) or directly from the modalities 106, 110, 112 (e.g., without routing through controller 114). For example, the separate system can be a separate controller, server, image processor, or data processing device that is local to or remote from system 100.

The system 100 can sequentially interrogate multiple points within and/or with respect to eye 102, for example, by moving the eye 102 and/or focal point. For example, the patient, or a portion thereof, can be supported by a one-dimensional, two-dimensional, or three-dimensional translation stage for moving the eye 102 with respect to patient interface 104. Alternatively or additionally, the patient interface 104, or a portion or portions thereof, can be mounted on or supported by a one-dimensional, two-dimensional, or three-dimensional translation storage for moving the focal point defined by the patient interface 104 with respect to the eye 102. Alternatively or additionally, the patient interface 104 can include one or more adjustable optical components to move the focal point in one-dimension, two-dimensions, or three-dimensions, for example, movable mirrors (e.g., galvanometer scanners) and/or movable lenses that redirect a light path and/or change a location of the focal point for interrogating and detected light. In some embodiments, controller 114 can control movement of the eye 102 and/or focal point, for example, by sending signals to the respective translation stage and/or scanning mechanism. Alternatively or additionally, positioning of the focal point with respect to the eye 102 can be manually controlled, for example, based on operator input to a user interface 116 (e.g., via a joystick, via on-screen selection of a point on a displayed image of a sample, etc.).

In an exemplary operation of system 100, the first imaging modality 110 (e.g., employing interrogating light 132) can measure corneal geometry of eye 102, for example, as shown in corneal geometry measurement stage 130 of FIG. 1B. For example, the first imaging modality 110 can obtain a corneal pachymetry map of the cornea 146 of the eye 102 in stage 130. Based on the corneal geometry measurements, a lateral point 136 having an extremum of the measured corneal geometry (e.g., minimum thickness or maximum curvature) can be identified, for example, as shown in extremum point identification stage 134 of FIG. 1B. For example, the thinnest corneal point (TCP) can be automatically determined (e.g., via controller 114) via software analysis in a manner similar to conventional processes. Based on the identified extremum point 136, the system 100 can define a focal test region 140 surrounding and including the extremum point 136 (e.g., such that all measurement points are within a first predetermined distance (e.g., 2-mm) from the identified extremum point).

In the Brillouin measurement stage 138 of FIG. 1B, multiple measurements can then be obtained via the Brillouin spectroscopy measurement modality 106 (e.g., employing interrogating light 150 at a different wavelength than interrogating light 132) for a plurality of lateral measurement points 142 localized within the focal test region 140 (e.g., a grid array of lateral points). In some embodiments, one of the measurement points can be the indicated extremum point 136. During the Brillouin measurement stage 138, the focal point can be moved to one of a plurality of lateral measurement points 142 (e.g., at a respective x-y location with respect to a front of the cornea 146), and to one of a plurality of depths 144 (e.g., at a respective z location with respect to the front of the cornea 146). After interrogating at the measurement location and acquiring the resulting Brillouin scattered light, the focal point can be moved (e.g., via movement of the eye 102 and/or by adjusting the patient interface) to the next of the plurality of lateral locations 142 and/or depths 144. Although particular locations and depths are illustrated in FIGS. 1B-1C, these locations and depths are exemplary for purposes of illustration. Other locations and/or depths (predetermined or otherwise) are also possible according to one or more embodiments. Moreover, although the locations and depths are illustrated in a regular arrangement (e.g., aligned to the x-y-z ordinate system, equal step size, etc.), other arrangements are also possible according to one or more embodiments.

In the calculation stage 152 of FIG. 1B, the Brillouin spectroscopy measurements from the focal test region 140 can be combined and/or processed to calculate a Brillouin Focality Index (BFI), which can provide enhanced SKC detection by focusing the Brillouin measurements on the most relevant corneal area (e.g., data obtained within a specific corneal depth in close proximity to the point of measured corneal geometry extremum). In particular, the BFI uses data from the focal test region to better differentiate normal corneas from those with SKC, thereby providing improved SKC detection. In some embodiments, the BFI can be calculated using only Brillouin spectrometry measurements obtained for points within 2-mm of the identified extrema point (e.g., TCP) and for points in the anterior stroma to a depth of 200-μm or 40% of the corneal thickness. For example, the BFI can be calculated based on a minimum Brillouin shift measured at a point within a second predetermined distance (e.g., 1-mm) from the identified extrema point (e.g., TCP), a maximum Brillouin shift measured at a point within the focal test region, and/or a plateau length derived from the measured Brillouin shifts at different depths. In some embodiments, a determination that the eye 102 exhibits SKC is made in response to the BFI being outside of a predetermined range, for example, a minimum Brillouin shift being less than a predetermined value. In some embodiments, controller 114 or a separate data processing system (not shown) can create the BFI and/or make the diagnosis or determination based on the BFI.

In addition to the refractive error to be corrected, all laser vision correction (LVC) treatments (LASIK, PRK, KLEx) use corneal pachymetry (thickness) and corneal curvature data as treatment input to program and complete laser treatment. The corneal mechanical data provided by the corneal analysis system 100 can allow the development of patient specific (e.g., customized) treatment. In some embodiments, the corneal analysis system can be modified to measure both corneal geometries, such that a single device measuring corneal thickness, curvature, and mechanics can provide all data necessary for refractive surgery screening as well as the performance of customized laser vision correction surgery for refractive error and/or customized corneal cross-linking treatments for keratoconus based on the patient's unique corneal mechanical profile.

For example, FIG. 1C illustrates such a combined corneal analysis system 160, where corneal geometry measurement module 108′ includes a third imaging modality 162 in addition to the first imaging modality 110. While the first imaging modality 110 may measure a first corneal geometry of the eye 102 (e.g., one of the thickness and the curvature), the second imaging modality 162 can be configured to obtain a measurement of a second corneal geometry of the eye 102 (e.g., the other of the thickness and the curvature). For example, when the first imaging modality 110 provides pachymetry (e.g., via OCT), the second imaging modality 162 can provide keratometry (e.g., via a Placido ring imager) either before or after the Brillouin spectroscopy measurements. Components for and operation of the system 160 may otherwise be similar to that described above for system 100 of FIG. 1A.

Referring to FIG. 1D, an exemplary diagnostic device 176 according to the configuration of corneal analysis system 160 is shown. In the illustrated example, the device 176 combines features of a motion-tracking Brillouin unit 170 (e.g., capable of measuring corneal thickness and Brillouin shifts) and a Placido ring imager 174 (e.g., capable of measuring corneal curvature) into a single unit 172. For example, the process of integrating Placido analysis with Brillouin and OCT of unit 170 can be accomplished through the addition of Placido rings surrounding the Brillouin/OCT imaging source. In some embodiments, the device 176 can further include a patient positioning means 178 for positioning the eye and/or supporting the head of the patient during the analysis. In some embodiments, the combination of mechanical data (Brillouin imaging), thickness data (OCT pachymetry), and curvature (Placido imaging) data can provide a comprehensive analysis of the patient's corneal profile that allows for customized laser vision correction for patients with normal corneal profiles and customized keratoconus management for patients with keratoconus, all based on the primary driver for outcomes-corneal mechanics derived from Brillouin imaging. In addition to its value in refractive surgery practices, such a combined device could also be used for youth vision screenings or in optometric practices as a noninvasive standard of care for new patient evaluations.

### Brillouin Spectroscopy Measurement Modality Configurations

FIG. 2A illustrates an exemplary setup 200 for a Brillouin spectroscopy measurement modality. A collimated light beam can be generated by a collimating lens 212 set in front of narrow linewidth Brillouin light source 214 (e.g., a laser locked to an absorption line of atomic gas cell or a broadband laser coupled with an appropriate high finesse filter). The collimated light beam can be directed from collimating lens 212 through a polarizer 210 (e.g., a linear polarizer), thereby resulting in a polarized light beam that is then reflected by polarization beam splitter 208 toward the eye. In the illustrated example, the reflected light from polarization beam splitter 208 can pass through a quarter-wave plate 216 to form the interrogating light beam. The interrogating light beam is Brillouin scattered by the eye, and the backward Brillouin scattered light is collected by the microscope optics (e.g., optics of the patient interface) and passes through the same quarter-wave plate 216, such that the polarization of the collected light allows it to pass through the polarization beam splitter 208 en route to the Brillouin detection arm 218 via reflecting element 206 (e.g., mirror) and coupling lens 202 (e.g., fiber collimator).

In some embodiments, a vapor cell 204 (e.g., atomic gas cell) can be disposed along the optical path between the polarization beam splitter 208 and the Brillouin detection arm 218. The vapor cell 204 can have one or more absorption lines (e.g., wavelengths) to remove stray interrogating light (e.g., having a wavelength of the light source 214) prior to detection by Brillouin detection arm 218. In some embodiments, the vapor cell 204 can exhibit a removal ratio of 104 or less (e.g., an absorption ratio greater than 10-3, for example, about 10-7) with respect to the light source wavelength. For example, the vapor cell 204 can be a rubidium vapor cell. Although FIG. 2A illustrates particular optical components and arrangements thereof for a Brillouin modality, fewer or different optical components and/or different arrangements of components are also possible according to one or more embodiments. Accordingly, embodiments of the disclosed subject matter are not limited to the details of FIG. 2A.

Due to the polarization properties of the cornea, the Brillouin signal as measured by the Brillouin spectroscopy measurement modality may be less than optimal in the corneal periphery. Thus, in some embodiment, the Brillouin spectroscopy measurement modality may be configured for non-polarized Brillouin imaging, which may allow for a more complete regional corneal biomechanical imaging (e.g., which may be useful for regional mechanical mapping in laser vision correction treatment algorithm development and application).

For proper Brillouin analysis of samples, Brillouin spectrometers generally require at least 60-dB spectral contrast to discriminate the Brillouin signal from much larger back-reflections, elastic scattering, and stray light. To achieve such a level of discrimination, Brillouin measurement systems can employ tandem Fabry-Perot interferometers, double-stage VIPA spectrometers, or single-stage VIPA spectrometer combined with other filters (e.g., vapor cells, Fabry-Perot etalons, etc.). However, the spectral extinction of the spectrometer may be less useful, or even useless, if the interrogating light source is not sufficiently narrow and clean (e.g., without any components within the Brillouin spectral region). Indeed, any incident light that is shifted from the main central peak of the interrogating light may be transmitted without filtering by the spectrometer apparatus, thereby obscuring or changing the Brillouin measurement. Narrow and clean light sources are generally easy to find in the visible range, since they typically employ solid-state lasers having narrow and pure transitions. Indeed, several conventional Brillouin instruments employ interrogating light at 532-nm or 660-nm, which are lines from Nd:Yag solid-state lasers. In contrast, for Brillouin measurements of the eye, wavelengths in the non-visible spectrum are used, for example, near infrared (e.g., at 780-nm). For such near infrared wavelengths, semiconductor lasers at typically available, but result in insufficiently narrow or unclean output.

Conventional solutions to “clean” the laser spectrum include Bragg gratings (effective at reducing amplified spontaneous emission (ASE)) and Fabry-Perot (FP) etalons (effective at suppressing side modes). However, the combined cost of such a light source can be high, and thus impractical for implementation in a clinical corneal analysis system. In contrast, in some embodiments, the Brillouin spectroscopy measurement modality can employ a broadband light source 222 in combination with a high finesse filter 224 as the Brillouin light source 214, as shown in FIG. 2B. For example, the light source 222 can be broadband laser (e.g., semiconductor laser) emitting light 226 having a large bandwidth (e.g., at least 1-GHz, such >>1-GHz), and the filter 224 can be a fiber-based Fabry-Perot (FFP) filter providing a finesse of at least 100 (e.g., ≥1,000 or ≥10,000) and/or an extinction of at least 60-dB (e.g., ˜70-dB to 80-dB, inclusive).

FFP filters are similar in principle to traditional FP etalons, but FFP filters can offer much higher finesse (e.g., <100 for FP etalons) and thus greater extinction (e.g., ˜30-dB for FP etalons). As a result, traditional FP etalons can only be used as additional clean up filters for Brillouin applications. Moreover, the free-spectral range (FSR) of a filter is given by the linewidth of the filter times the finesse. The desired linewidth for interrogating light 228 is dictated by the Brillouin spectrum characteristics, for example, about 100-MHz. An FP etalon may typically have a FSR less than 10-GHz, potentially providing several lines disturbing the spectral region dedicated to Brillouin spectra, while FFP filters can provide a single line in the Brillouin spectral region. Given these characteristics, in some embodiments, a suitable light source for Brillouin microscopy can be provided by a broadband laser source combined with an FFP filter. Strong restrictions on bandwidth (e.g., >>1-GHz) or extinction (both ASE and Side mode suppression ration <30-dB) are not required for the laser source because the FFP filter will carve from its spectrum a single line with desired linewidth (e.g., ˜100-MHz) and large extinction (e.g., >60-dB).

Alternatively or additionally, in some embodiments, the Brillouin spectrometer detection arm 218 can employ a single spectral dispersion stage (e.g., Virtually Imaged Phased Array (VIPA)), for example, by employing an optimized atomic vapor filtering element. For example, FIG. 2C illustrates an example of such a detection arm configuration, where light 230 from the eye is directed to a single VIPA 232. The output of the VIPA 232 can be directed to a first lens 234, which can have a same focal length as coupling lens 202 (e.g., f=200-mm). The output of the first lens 234 can be directed to a second lens 236, which can have a smaller focal length than first lens 234 (e.g., f=40-mm). The output of the second lens 236 can be directed to a filter 238, for example, an atomic vapor filtering element. The output of the filter 238 can be directed to a pair of imaging lenses 240, 244 separated by a confocal aperture 242. In some embodiments, the focal lengths of the imaging lenses may be different, for example, with the focal length of the first imaging lens 240 (e.g., f=30-mm) being smaller than the focal length of the second imaging lens 244 (e.g., f=40-mm). The output from imaging lens 244 can be directed to a detector 246, for example, an electron-multiplying charge-coupled device (EM-CCD). In some embodiments, such a configuration for the detection arm 218 can improve spectrometer resolution and throughput to achieve equivalent signal-to-noise ratio (SNR) and spectral contrast while reducing acquisition time by five times (5×), for example, as compared to conventional Brillouin spectroscopy detection arm configurations.

In some embodiments, the corneal analysis system, or at least the Brillouin modality thereof, can be configured for relatively fast measurement acquisition (e.g., <5-minutes, for example, ≤3-minutes, or even≤1-minute for a complete corneal scan, as compared to >10-20 minutes for prior systems). In some embodiments, the time to capture all Brillouin spectroscopy measurement data for a focal screening scan needed to calculate the BFI can be less than or equal to 30-seconds. In some embodiments, the Brillouin imaging process can utilize a motorized translational stage controlled by computer software for automated location determination in 3-dimensions and/or a manual joystick for gross adjustments as needed for patient alignment.

In some embodiments, the Brillouin imaging performed by the corneal analysis system can be at a spatial resolution (X×Y×Z) of less than or equal to 5-μm×5-μm×100-μm, for example, less than or equal to 2-μm×2-μm×18-μm. Alternatively or additionally, the Brillouin imaging performed by the corneal analysis system can be at a spectral resolution of less than or equal to 800-MHz, for example, less than or equal to 250-MHz. Alternatively or additionally, the Brillouin imaging performed by the corneal analysis system can employ a spectral extinction of less than or equal to 72-dB, for example, less than or equal to 70-dB. Alternatively or additionally, the Brillouin imaging performed by the corneal planning system can offer a modulus sensitivity (ΔM′/M′) of at least 0.1%. Alternatively or additionally, the corneal planning system can perform spectrum integration of the Brillouin spectroscopy data in a time less than or equal to 50-ms, for example, less than or equal to 10-ms. Alternatively or additionally, the corneal planning system can perform an axial scan (e.g., at a single lateral measurement point) of the Brillouin imaging in less than or equal to 3-seconds for at least 60 points (e.g., depths), for example, less than or equal to 1-second for at least 100 points (e.g., depths). Alternatively or additionally, the corneal planning system can perform the Brillouin imaging for different lateral measurement points with a lateral positioning delay of less than or equal to 15-seconds, for example, less than or equal to 1.5 seconds. Alternatively or additionally, the corneal planning system can perform the Brillouin imaging for at least 50 lateral points (including different axial depths) in a time of less than or equal to 20-minutes, for example, less than or equal to 5-minutes, less than or equal to 3-minutes, or even less than or equal to 1-minute.

In some embodiments, image processing by the corneal planning system can utilize a high-finesse Fabry-Perot filter as a band-pass to filter outside modes optically, which can enable automatic Brillouin peak fitting combined with convolutional analysis between sequential OCT spectra to automatically determine the cornea surface. This process can allow for an increase in depth points (e.g., at least 100 depth points) and depth resolution (e.g., 20-μm or less). Alternatively or additionally, an algorithm can be used to determine the slope of the Brillouin depth profile, for example, to more accurately segment the different regions of the axial scan in an automated fashion.

### Optical System Configurations

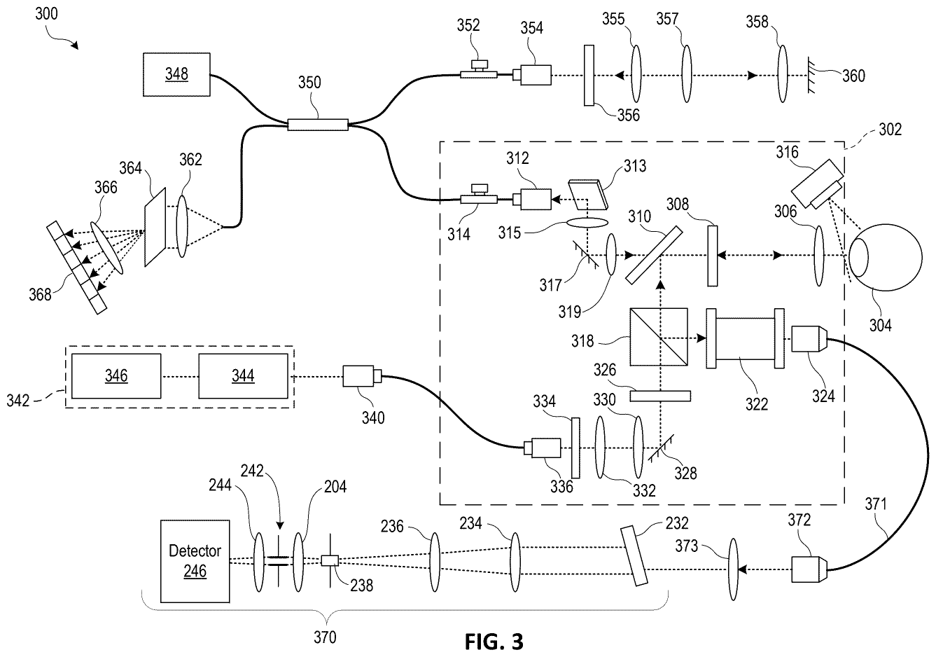

Referring to FIG. 3, a corneal analysis system 300 for determining a patient's eye 304 exhibits subclinical keratoconus is shown. In the illustrated example, the system 300 includes a patient interface 302, a Brillouin modality for corneal modulus measurement, an optical coherence tomography (OCT) device for axial tracking and corneal thickness measurement, and a single-lens imaging system 316 for in-plane pupil tracking. In some embodiments, the components of the patient interface 302 can be mounted on a breadboard sitting on an ophthalmic slit lamp frame, which can be moved in three dimensions, for example, via a joystick. To provide spatially-resolved Brillouin shifts across the cornea, the Brillouin modality can have a confocal microscopy configuration. The light source 342 of the Brillouin modality can be a broadband light source 346 (e.g., emitting broadband light at 780-nm) in combination with a fiber-based Fabry Perot filter 344. The light from the source 342 can be coupled into a polarization-maintaining fiber via coupler 340 and directed to the patient interface 302.

In the patient interface 302, after passing through a polarizer 334, the output beam from fiber collimator 336 can be enlarged by a telescope made up by two lenses 332 (e.g., f=20-mm) and 330 (e.g., f=75-mm), for example, to get an effective 0.1 numeric aperture (NA) on lens 306 (e.g., f=50-mm) via reflecting element 328 (e.g., mirror), half-wave plate 326, polarization beam splitter (PBS) 318, dichroic mirror 310, and quarter-wave plate 308. In some embodiments, lens 306 can be mounted on a translational stage (not shown) to enable axial scanning through the corneal depth.

To detect the Brillouin frequency shift, AvB, the scattered light from the eye 304 can be collected by the lens 306, passed through quarter-wave plate 308, reflected by dichroic mirror 310, and then redirected by PBS 318 to pass through gas cell 322, which can remove the unshifted frequency. Objective lens 324 (e.g., 4×) can be used to couple the light exiting gas cell 322 into a fiber 371 (e.g., multimode fiber), which directs the light to Brillouin spectroscopy detection arm 370 via objective lens 372 (e.g., D=1.31-mm) and coupling lens 373 (e.g., f=200-mm). In the illustrated example, the Brillouin spectroscopy detection arm 370 has a configuration similar to that of detection arm 218 of FIG. 2C.

In some embodiments, to track axial movement of the measured point, the sampling arm of a frequency-domain OCT can share the same optical path with the light source 346 through dichroic mirror 310. In some embodiments, the light source 348 of the OCT can be a superluminescent diode (SLD) with a bandwidth of 50-nm and a central wavelength of about 840-nm. For example, the OCT can have an axial resolution of 6.2-μm. In some embodiments, the power of the OCT light source 348 can be equally split into reference and sampling arms by fiber coupler 350. For example, the collimated beam diameters at the outputs of the fiber coupler 350 can be about 1.31-mm. The reference arm of the OCT can include a polarization controller 352, a fiber coupler 354, a neutral density filter 356, a train of lenses 355, 357, 358, and a reflecting element 360 (e.g., mirror), and the sampling arm of the OCT can include a polarization controller 314, a fiber coupler 312, galvanometer scanner 313, reflecting element 317 (e.g., mirror), pair of lenses 315, 319, dichroic mirror 310, quarter-wave plate 308, and lens 306. In some embodiments, the focal lengths of lens 306 and the corresponding lens 358 can be the same, for example, about 50-mm (e.g., corresponding to a transverse resolution of about 40.8-μm and a depth of focus (DOF) of about 3.1-mm).

In some embodiments, after focusing light from OCT light source 348 onto the cornea of eye 304, the scattered light can be interfered with the reflection from the reference arm and analyzed by a separate spectrometer. For example, the spectrometer can be formed by a first achromatic lens 362 (e.g., f=45-mm), a volume phase holographic grating (VPHG) 364 (e.g., 1800-lp/mm), a second achromatic lens 366 (e.g., f=100-mm), and a line camera 368. Within the spectrometer, incident light can be diffracted by the VPHG 364 and imaged onto line camera 368 via the second achromatic lens.

Besides potential axial movement during a scan, the eye could also exhibit lateral movement. In some embodiments, imaging system 316 can be placed next to the objective lens 306 to monitor this lateral movement, for example, by tracking the pupil of the eye 304. In some embodiments, imaging system 316 can have its own light source, for example, a light-emitting diode (LED) having a central wavelength of about 970 nm. For example, the illumination power on the whole eye 304 can be about 2-mW, and the exposure time of the camera of imaging system 316 can be about 20-ms. In some embodiments, a long-pass filter can be provided before the camera of imaging system 316, for example, to reject light from the Brillouin light source 346 and/or keep at least part of the light from the OCT light source 348, for example, for use in annotating measured points. In some embodiments, a ratio of distance/pixel can be calculated by imaging a standard grid on the focal plane, and a pupil tracking algorithm can employ binarization and edge detection.

The combination of Brillouin modality with one or more imaging modalities for corneal geometry measurement and/or tracking can be realized in many different configurations, and the system 300 of FIG. 3 represents only one of such configurations. However, embodiments of the disclosed subject matter are not limited to the configuration specifically described for system 300. Rather, other configurations for corneal analysis systems employing Brillouin spectroscopy are also possible according to one or more contemplated embodiments.

### Corneal Analysis Methods

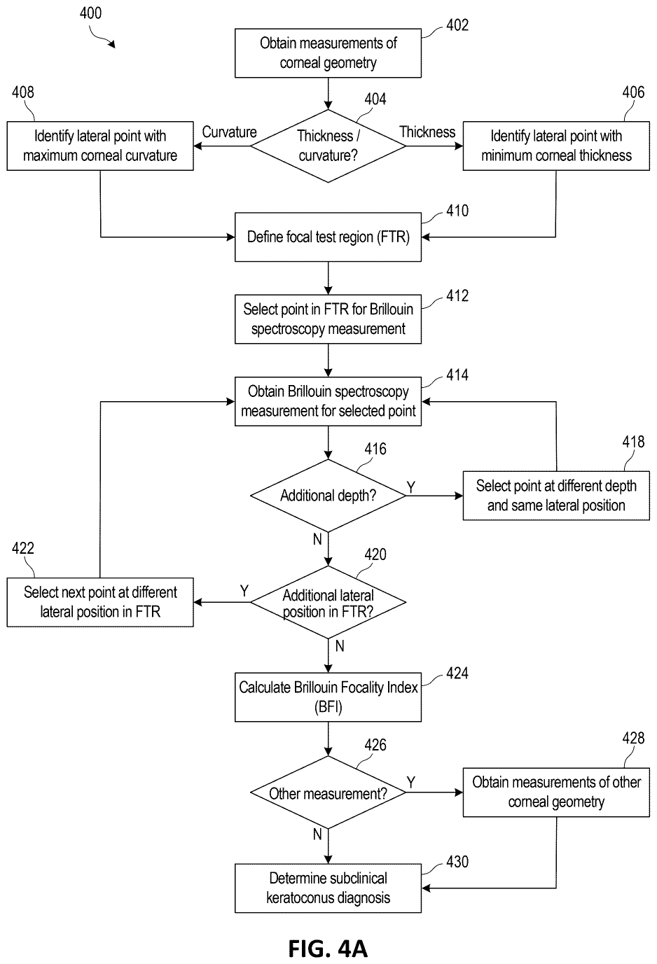

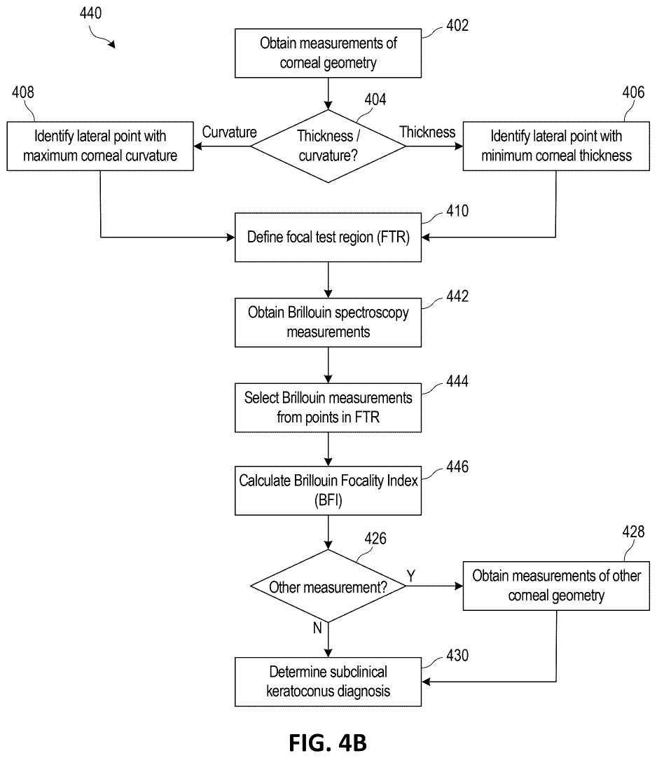

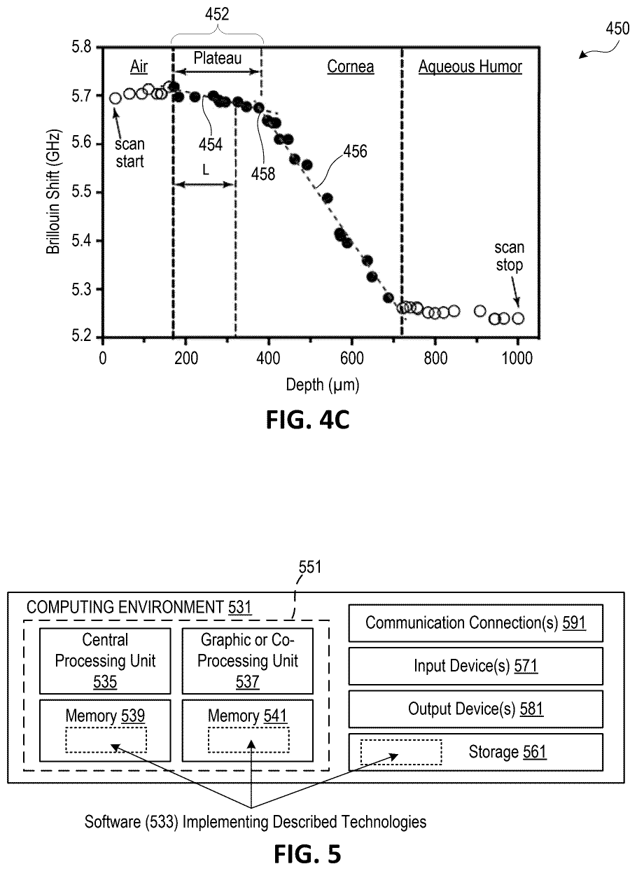

FIG. 4A illustrates aspects of a method 400 for detection of SKC via Brillouin spectroscopy measurements. The method 400 can initiate at process block 402, where measurements of one or more corneal geometry characteristics can be obtained, for example, either corneal thickness or curvature. In some embodiments, the measurement of corneal thickness can be via optical interrogation, for example, OCT, Scheimpflug imaging, slit-scanning technology, laser-scanning confocal microscopy, specular microscopy, optical biometry, etc. In some embodiments, the optical interrogation to measure corneal geometry can be performed by a same imaging modality in a corneal analysis system used to track the eye (e.g., with respect to depth) during Brillouin spectroscopy measurements. Alternatively or additionally, in some embodiments, the measurement of corneal thickness can be via a non-optical method, such as ultrasound pachymetry. In some embodiments, the measurement of corneal curvature can be via optical interrogation, for example, Placido ring imager, or via other keratometry device.

If corneal thickness was measured in process block 402, the method 400 can proceed via decision block 404 to process block 406, where a lateral point of the cornea having a minimum thickness (e.g., thinnest corneal point (TCP)) can be identified. Alternatively, if corneal thickness was measured in process block 402, the method can proceed via decision block 404 to process block 408, where a lateral point of the cornea having a maximum curvature (e.g., maximum curvature point (MCP)) can be identified. The method 400 can proceed from either process block 406 or process block 408 to process block 410, where a focal test region (FTR) can be defined based on the identified extremum point (e.g., either TCP or MCP). In some embodiments, the FTR can be defined as a two-dimensional lateral region (e.g., in the x-y plane) surrounding and including the identified extremum point, for example, such that each subsequent measurement point within the FTR is no more than a predetermined lateral distance (e.g., 2-mm or less) from the identified extremum point.

The method 400 can proceed to process block 412, where a first measurement point within the FTR can be selected for Brillouin spectroscopy measurements. In some embodiments, the first measurement point may coincide with the identified extremum point (e.g., either TCP or MCP). The method 400 can then proceed to process block 414, where a Brillouin spectroscopy measurement can be obtained for the selected measurement point within the FTR of the eye. In some embodiments, the obtaining of process block 414 can include concurrently tracking lateral location and depth of the measurement point and/or movement of the patient's eye (e.g., via one or more optical imaging modalities) and correlating the Brillouin spectroscopy measurement with a corresponding location with respect to a patient's eye. For example, in some embodiments, the system 300 of FIG. 3 can be used to direct light to and collect scattered Brillouin light from the patient's eye to perform a Brillouin spectroscopy measurement, as well as to provide tracking.

The method 400 can proceed to decision block 416, where it is determined if additional depths should be interrogated. For example, in some embodiments, Brillouin spectroscopy measurements are obtained for different depths (e.g., an axial scan for at least 100 different depths) with respect to a patient's eye at a same lateral measurement point. If additional depths are desired, the method 400 can proceed to process block 418, where the focal point for the Brillouin modality is advanced to the next depth, for example, by moving one or both of the focal point and the eye with respect to each other along an axial direction. After moving to the next depth, the method 400 can proceed from process block 418 to process block 414, where Brillouin spectroscopy measurement(s) are obtained at the new depth for the same lateral position.

The method 400 can proceed to decision block 420, where it is determined if additional lateral points should be interrogated. For example, in some embodiments, Brillouin spectrometry measurements are obtained at different lateral positions within the FTR. For example, in some embodiments, about 50 points can be measured at different lateral locations within the FTR. If additional measurement points are desired, the method 400 can proceed to process block 422, where the focal point for the Brillouin modality can be advanced to the next lateral measurement point, for example, by laterally moving one or both of the focal point and the eye with respect to each other (e.g., across a front surface of the cornea). In some embodiments, a next lateral measurement point can be instructed by an operator of the system (e.g., via joystick or manual selection on a displayed image of the eye). After moving to the next lateral measurement point, the method can return to process block 414, where a Brillouin spectroscopy measurement can be obtained at the new lateral measurement point.

If all desired lateral points within the FTR have been evaluated, the method 400 can proceed from decision block 420 to process block 424, where the Brillouin spectrometry measurements for the different lateral positions within the FTR can be used to calculate a Brillouin Focality Index (BFI). In some embodiments, the BFI can be based on Brillouin measurements at the identified extremum point and for points within the FTR (e.g., within 2-mm of the identified extremum point) and in the anterior stroma to a depth of 200-μm or 40% of corneal thickness. For example, the BFI can be calculated based on a minimum Brillouin shift value within 1-mm of the identified extremum point, a maximum Brillouin shift value within the FTR (e.g., to provide an indication of biomechanical heterogeneity or to normalize the minimum value, for example, as a ratio), and/or an axial plateau length for the Brillouin measurements. In some embodiments, the BFI can be calculated by a controller of a corneal analysis system (e.g., controller 114) or a separate data processing system.

In some embodiments, the plateau length can be measured through an objective, multi-step process that uses linear fitting to analyze the Brillouin depth profile and identify the boundaries of the plateau region. Referring to FIG. 4C, to objectively identify a plateau region 452, the depth profile 450 for a particular lateral measurement point can be fit with a first linear fit 454 of low shift change per depth (the “plateau” region) and a second linear fit 456 of high shift change per depth (the “slope” region). The intersection 458 of these two linear fits 454, 456 marked a posterior end of the plateau region 452. The anterior end of the plateau region 452 can be determined by the tail of the second linear fit 456 (e.g., the slope region) and a thickness of the cornea, for example, as measured by the corneal geometry measurement modality (e.g., OCT).

Returning to FIG. 4A, the method 400 can proceed to decision block 426, where it can be determined if other measurements are desired, for example, measurements of a different corneal geometry characteristic different from that in process block 402. If such measurements are desired, the method 400 can proceed to process block 428, where measurements of another corneal geometry characteristic (as compared to that in process block 402) can be obtained, for example, the other of corneal thickness or curvature. In some embodiments, the corneal geometry measurement can be performed by another optical imaging modality of the corneal analysis system. In some embodiments, the measurement of corneal curvature can be via optical interrogation, for example, Placido ring imager, or via other keratometry device. In some embodiments, the measurement of corneal thickness can be via optical interrogation, for example, OCT, Scheimpflug imaging, slit-scanning technology, laser-scanning confocal microscopy, specular microscopy, optical biometry, etc. Alternatively or additionally, in some embodiments, the measurement of corneal thickness can be via a non-optical method, such as ultrasound pachymetry.

The method 400 can proceed to process block 430, where a determination that the eye exhibits SKC can be made responsive to the BFI. For example, a determination of SKC can be made when the BFI is outside a predetermined range or less than a predetermined threshold. In some embodiments, the determination of SKC can be made by a controller of the measurement system (e.g., system 300, or a controller 114 of system 100 or system 160), or a separate data processing system. Alternatively, in some embodiments, the determination of SKC is made by an operator of the measurement system or a separate data processing system, or by a clinician or physician that receives the BFI.

Although blocks 402-430 of method 400 have been described as being performed once, in some embodiments, multiple repetitions of a particular block may be employed before proceeding to the next decision block or process block. In addition, although blocks 402-430 of method 400 have been separately illustrated and described, in some embodiments, blocks may be combined and performed together (simultaneously or sequentially). Moreover, although FIG. 4A illustrates a particular order for blocks 402-430, embodiments of the disclosed subject matter are not limited thereto. Indeed, in certain embodiments, the blocks may occur in a different order than illustrated or simultaneously with other blocks.

In some embodiments, method 400 can include steps or other aspects not specifically illustrated in FIG. 4A. Alternatively or additionally, in some embodiments, method 400 may comprise only some of blocks 402-430 of FIG. 4A. For example, in some embodiments, method 400 can include some or all of blocks 402, 406, 408, 410, and 424 (e.g., with or without obtaining other corneal geometry measurements 428), e.g., to perform a data processing method for previously obtained Brillouin spectroscopy measurements. Alternatively or additionally, in some embodiments, method 400 can include some or all of blocks 412-422, e.g., to perform a data acquisition method that obtains Brillouin spectroscopy measurements for subsequent processing.

In the illustrated example of FIG. 4A, Brillouin spectroscopy measurements are only obtained for the focal test region. However, in some embodiments, Brillouin spectroscopy measurements can also be obtained outside the focal test region (e.g., for a larger portion of the eye), and the BFI can be calculated using only measurements from points within the focal test region. In such embodiments, the corneal geometry measurements and/or the Brillouin spectroscopy measurements can be obtained by the same or separate systems, and the data processed after acquisition to define the focal test region and select data from measurement points within the focal test region.

For example, FIG. 4B illustrates aspects of another method 440 for detection of SKC via Brillouin spectroscopy measurements. Blocks 402-410 and 426-430 of method 440 can be similar to blocks 402-410 and 426-430 of method 400, as described above. Method 440 can proceed to process block 442, where Brillouin spectroscopy measurements can be obtained for a plurality of lateral measurement points and corresponding axial depths. For example, the obtained measurements in process block 442 can be for points outside the focal test region (FTR), as well as points within the FTR. The method 440 can proceed to process block 444, where only the Brillouin spectroscopy measurements for points within the FTR are selected and subsequently used in process block 446 to calculate the BFI, for example, in a manner similar to process block 424 of method 400, as described above.

Although blocks 402-410, 426-430, and 442-446 of method 440 have been described as being performed once, in some embodiments, multiple repetitions of a particular block may be employed before proceeding to the next decision block or process block. In addition, although blocks 402-410, 426-430, and 442-446 of method 440 have been separately illustrated and described, in some embodiments, blocks may be combined and performed together (simultaneously or sequentially). Moreover, although FIG. 4B illustrates a particular order for blocks 402-410, 426-430, and 442-446, embodiments of the disclosed subject matter are not limited thereto. Indeed, in certain embodiments, the blocks may occur in a different order than illustrated or simultaneously with other blocks. In some embodiments, method 440 can include steps or other aspects not specifically illustrated in FIG. 4B. Alternatively or additionally, in some embodiments, method 440 may comprise only some of blocks 402-410, 426-430, and 442-446 of FIG. 4B.

### Computer Implementation Examples

FIG. 5 depicts a generalized example of a suitable computing environment 531 in which the described innovations may be implemented, such as but not limited to controller 114, method 400, and/or method 440. The computing environment 531 is not intended to suggest any limitation as to scope of use or functionality, as the innovations may be implemented in diverse general-purpose or special-purpose computing systems. For example, the computing environment 531 can be any of a variety of computing devices (e.g., desktop computer, laptop computer, server computer, tablet computer, etc.).

With reference to FIG. 5, the computing environment 531 includes one or more processing units 535, 537 and memory 539, 541. In FIG. 5, this basic configuration 551 is included within a dashed line. The processing units 535, 537 execute computer-executable instructions. A processing unit can be a central processing unit (CPU), processor in an application-specific integrated circuit (ASIC), or any other type of processor (e.g., hardware processors, graphics processing units (GPUs), virtual processors, etc.). In a multi-processing system, multiple processing units execute computer-executable instructions to increase processing power. For example, FIG. 5 shows a central processing unit 535 as well as a graphics processing unit or co-processing unit 537. The tangible memory 539, 541 may be volatile memory (e.g., registers, cache, RAM), non-volatile memory (e.g., ROM, EEPROM, flash memory, etc.), or some combination of the two, accessible by the processing unit(s). The memory 539, 541 stores software 533 implementing one or more innovations described herein, in the form of computer-executable instructions suitable for execution by the processing unit(s).

A computing system may have additional features. For example, the computing environment 531 includes storage 561, one or more input devices 571, one or more output devices 581, and one or more communication connections 591. An interconnection mechanism (not shown) such as a bus, controller, or network interconnects the components of the computing environment 531. Typically, operating system software (not shown) provides an operating environment for other software executing in the computing environment 531, and coordinates activities of the components of the computing environment 531.

The tangible storage 561 may be removable or non-removable, and includes magnetic disks, magnetic tapes or cassettes, CD-ROMs, DVDs, or any other medium which can be used to store information in a non-transitory way, and which can be accessed within the computing environment 531. The storage 561 can store instructions for the software 533 implementing one or more innovations described herein.

The input device(s) 571 may be a touch input device such as a keyboard, mouse, pen, or trackball, a voice input device, a scanning device, or another device that provides input to the computing environment 531. The output device(s) 581 may be a display, printer, speaker, CD-writer, or another device that provides output from computing environment 531.

The communication connection(s) 591 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, radio-frequency (RF), or another carrier.

Any of the disclosed methods can be implemented as computer-executable instructions stored on one or more computer-readable storage media (e.g., one or more optical media discs, volatile memory components (such as DRAM or SRAM), or non-volatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or any other such network) using one or more network computers.

For clarity, only certain selected aspects of the software-based implementations are described. Other details that are well known in the art are omitted. For example, it should be understood that the disclosed technology is not limited to any specific computer language or program. For instance, aspects of the disclosed technology can be implemented by software written in C++, Java™, Python®, and/or any other suitable computer language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means. In any of the above-described examples and embodiments, provision of a request (e.g., data request), indication (e.g., data signal), instruction (e.g., control signal), or any other communication between systems, components, devices, etc. can be by generation and transmission of an appropriate electrical signal by wired or wireless connections.

### Fabricated Examples and Experimental Results

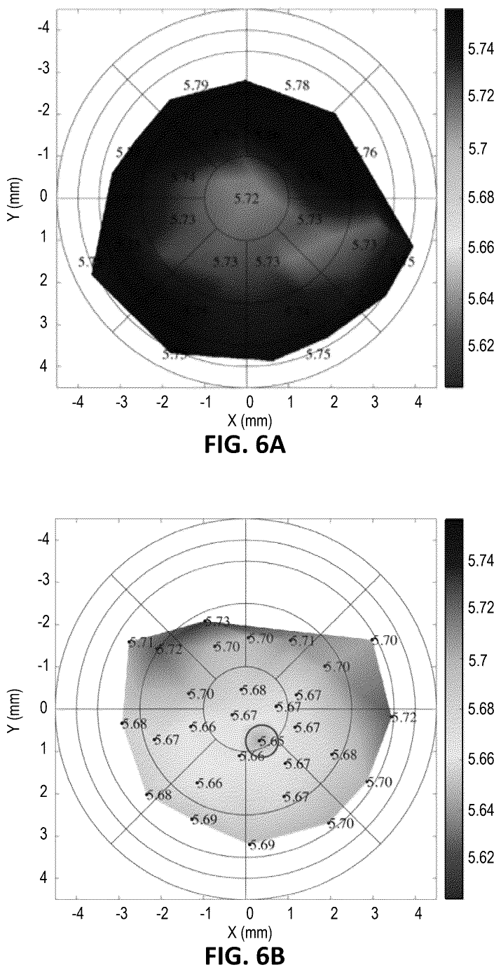

Motion-tracking Brillouin spectroscopy (MTB) was performed on normal eyes (control) and eyes exhibiting subclinical keratoconus (SKC). Normal eyes showed homogenously stiffer corneas, as exemplified in the map obtained by MTB in FIG. 6A. In contrast, SKC eyes showed significant focal weakening, as exemplified in the map obtained by MTB in FIG. 6B. This data further demonstrates that a focally reduced modulus is the initial event in development of SKC and thus can provide an early indicator of SKC. Compared with conventional technologies, MTB also exhibited superior performance in detecting SKC. For example, the eye evaluated by MTB to generate the map of FIG. 6B was also evaluated by Scheimpflug imaging, Pentacam Enhanced Ectasia Display, OCT, and Corvis. The basic Scheimpflug image showed elevation-derived anterior curvature, regional pachymetry, and anterior and posterior surface elevation images. There was subtle asymmetric anterior curvature without any notable abnormalities in pachymetry or elevation maps, thus indicating a normal cornea based on basic Scheimpflug tomography. The Pentacam Enhanced Ectasia Display showed multiple automated metrics designed specifically for subclinical keratoconus detection, with primary output including the BAD-D, ARTmax, and Back Elevation. There were no abnormalities in any index thus indicating a normal cornea based on the Pentacam Enhanced Ectasia metrics. The OCT provided images of total and epithelial thicknesses with multiple automated metrics. Again, there were no abnormalities in either pachymetry or epithelial maps, thus indicating a normal cornea based on OCT. The primary output from the Corvis corneal biomechanical device also indicated no abnormalities in any of the reported Corvis metrics (CBI, TBI, PRFI, BAD-D indices), thus indicating a normal cornea based on Corvis biomechanics. In contrast, the MTB analysis of the same eye shows focal weakening that is clearly notable in the center of the cornea (5.65 GHz, circle in FIG. 6B), which clearly indicates focal corneal weakening in this subclinical keratoconus eye.

To evaluate 3-dimensionally (3-D) localized mechanical differences that characterize SKC and to determine their relative ability to effectively differentiate the normal cornea from eyes with SKC and clinically manifest KC, normal controls (Controls), patients with SKC, and patients with clinically manifest keratoconus KC were evaluated (n=10/group). MTB microscopy was used to characterize the mechanical properties (measured as the minimum Brillouin shift, corresponding to the softest/weakest corneal point) from the anterior, middle, and posterior corneal regions and the plateau region depth. Corneal thickness and location of the thinnest corneal point were determined by a commercially available optical coherence tomography (OCT) device (Avanti, Visionix, Inc.). As shown in Table 1, minimum Brillouin shift values were highest in the anterior cornea followed by middle followed by posterior regions in all groups, Brillouin shifts were higher in Controls followed by SKC followed by KC eyes in each region, and the length of the plateau region was greater in Controls than SKC or KC eyes.

TABLE 1
MTB Metrics for Controls, SKC, and KC Groups

| | Thinnest | | | | Plateau | Distance from TCP to |
|---|---|---|---|---|---|---|
| | Corneal | Minimum Brillouin Shift (GHz) | | | (% of corneal | Anterior Min. Brillouin |
| Group | Thickness (μm) | Anterior | Middle | Posterior | thickness) | Shift (mm) |
| Normal | 544.1 | 5.698 | 5.601 | 5.369 | 44.0 | 0.46 |
| SKC | 506.6 | 5.660 | 5.561 | 5.355 | 37.1 | 0.45 |
| KC | 468.1 | 5.642 | 5.538 | 5.346 | 34.2 | 1.12 |

As shown in Table 2, there were significant differences in minimum Brillouin Shift between Controls and SKC eyes in Anterior and Middle corneal regions, as well as Plateau length. Anterior Min Shift values best differentiated Control and SKC groups (AUROC=1), followed by Plateau length (0.98) and Middle Region Shift (0.94). Posterior Min Brillouin Shift was not significantly different and did not differentiate groups well. There were no differences between groups in the distance between the thinnest corneal point and the Anterior Min Brillouin Shift.

As shown in Table 3, there were significant differences in Min Brillouin Shift between Controls and KC eyes in all regions and Plateau length. Anterior Min Shift values and Plateau length equally differentiated Control and KC groups (AUROC=1), followed by Middle Min Shift (0.98) and Thinnest corneal point (0.94). Posterior Min Brillouin Shift performed better at discriminating between Controls and KC (as compared to between Controls and SKC) but still performed worse than other metrics. There were no differences between groups in the distance between the thinnest corneal point (TCP) and the Anterior Min Brillouin Shift.

TABLE 2
Receiver Operating Characteristics Curve Data - Control and SKC

| | | | | Sensi- | Speci- |
|---|---|---|---|---|---|
| | P | | | tivity | ficity |
| Variable | value | AUROC | Cut-off | (%) | (%) |
| Anterior - minimum | 0.0002 | 1 | <5.680 | 100 | 100 |
| Brillouin shift | | | | | |
| Plateau | 0.0003 | 0.98 | <40.32 | 90 | 100 |
| (% total) | | | | | |
| Middle - minimum | 0.0009 | 0.94 | <5.575 | 90 | 90 |
| Brillouin shift | | | | | |
| Thinnest corneal | 0.033 | 0.8 | <536.3 | 87.5 | 50 |
| point (TCP) | | | | | |
| Posterior - minimum | 0.0821 | 0.73 | <5.363 | 70 | 80 |
| Brillouin shift | | | | | |
| TCP to | 0.894 | 0.5188 | >0.4450 | 50 | 50 |
| minimum distance | | | | | |

TABLE 3
Receiver Operating Characteristics Curve Data - Control and KC

| | | | | Sensi- | Speci- |
|---|---|---|---|---|---|
| | P | | | tivity | ficity |
| Variable | value | AUROC | Cut-off | (%) | (%) |
| Anterior - minimum | 0.0002 | 1 | <5.672 | 100 | 100 |
| Brillouin shift | | | | | |
| Plateau | 0.0002 | 1 | <40.16 | 100 | 100 |
| (% total) | | | | | |
| Middle - minimum | 0.0003 | 0.98 | <5.574 | 100 | 90 |
| Brillouin shift | | | | | |
| Thinnest corneal | 0.009 | 0.94 | <510.7 | 90 | 90 |
| point (TCP) | | | | | |
| Posterior - minimum | 0.0082 | 0.85 | <5.363 | 90 | 80 |
| Brillouin shift | | | | | |
| TCP to | 0.99 | 0.5 | >0.4216 | 60 | 50 |
| minimum distance | | | | | |

Thus, there are significant, 3-D differences in corneal mechanics between Controls, SKC, and KC eyes. Anterior corneal regional differences best differentiate Control and SKC eyes. This work thus demonstrates these regional mechanical differences between controls and SKC in vivo and may be used to guide focal mechanical screening for SKC identification

To determine relative positions of the TCP and the location of the Anterior Corneal Minimum Brillouin Shift (A-Min), normal controls (Controls, n=40 eyes), eyes with SKC (SKC, n=42 eyes), and eyes with clinically manifest KC (KC, n=14 eyes) were evaluated. In particular, MTB microscopy was used to measure local Brillouin shift values from the anterior corneal plateau region, from which the location of the minimum Brillouin shift (corresponding to the softest/weakest corneal point) was determined. Regional pachymetry was obtained by a commercially available optical coherence tomography (OCT) device (Avanti, Visionix, Inc.), from which the location of the TCP was determined.

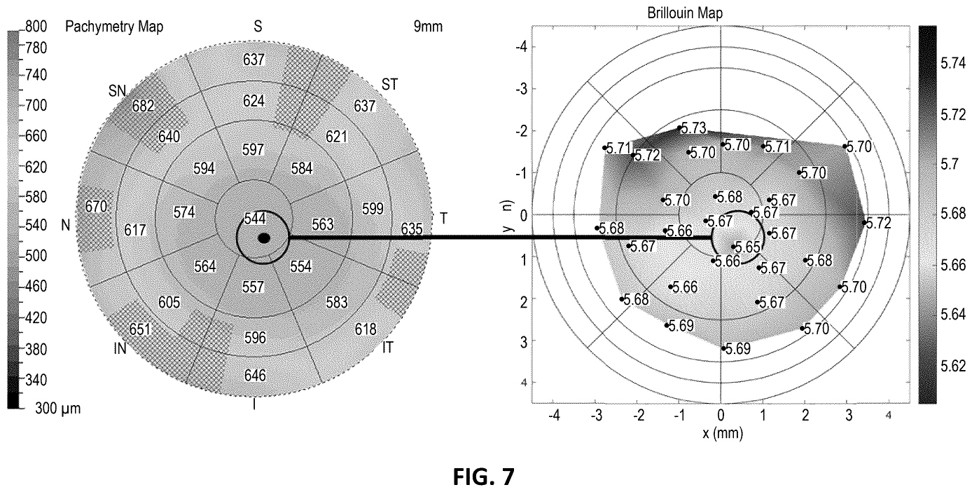

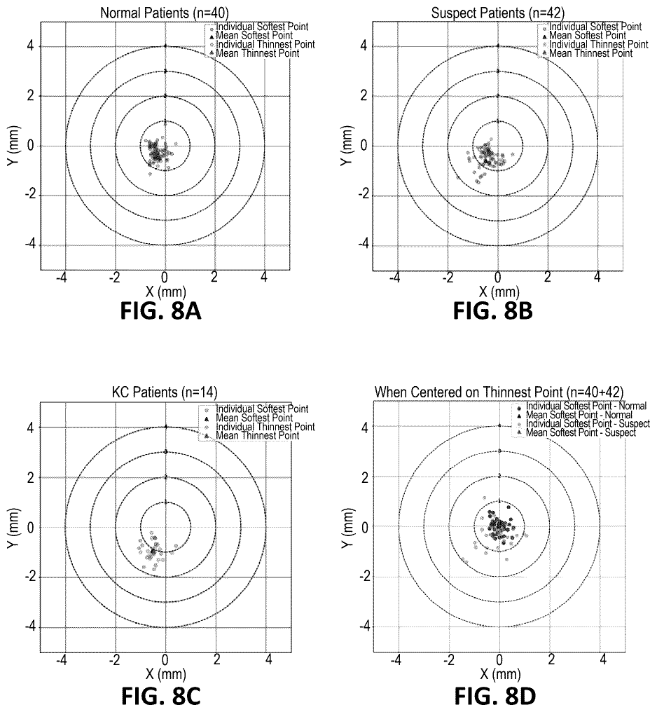

The Euclidian distance between these two points determined their relative co-localization. An example image is shown in FIG. 7. As summarized in Table 4, there were no significant differences between any of the groups in terms of co-localization distance or maximum co-localization distance. FIGS. 8A-8C shows the relative positions of the TCP and MTB A-Min for each cohort. There was tight overlap for TCL and A-Min values for each group. FIG. 8D shows the relative position of A-Min values for controls and SKC eyes when centered around TCP, showing near complete overlap between cohorts.

TABLE 4
MTB Metrics for Controls, SKC, and KC (P > 0.1)

| | TCP to A-MIN | | |
|---|---|---|---|
| | distance | Standard Deviation | Maximum distance |
| Group | (mm) | (mm) | (mm) |
| Normal | 0.34 | 0.16 | 0.69 |
| SKC | 0.43 | 0.26 | 0.93 |
| KC | 0.44 | 0.20 | 0.88 |

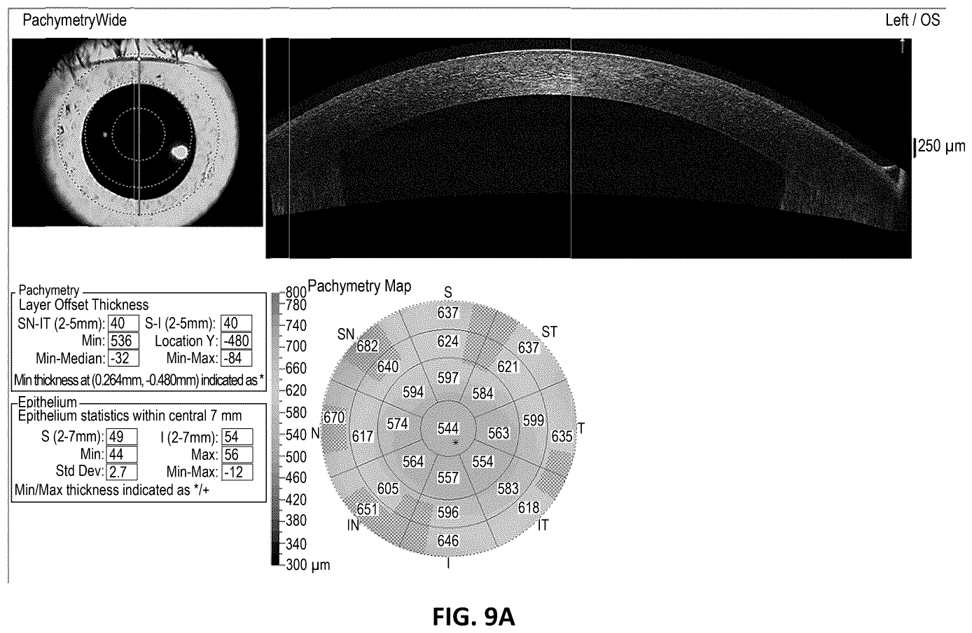

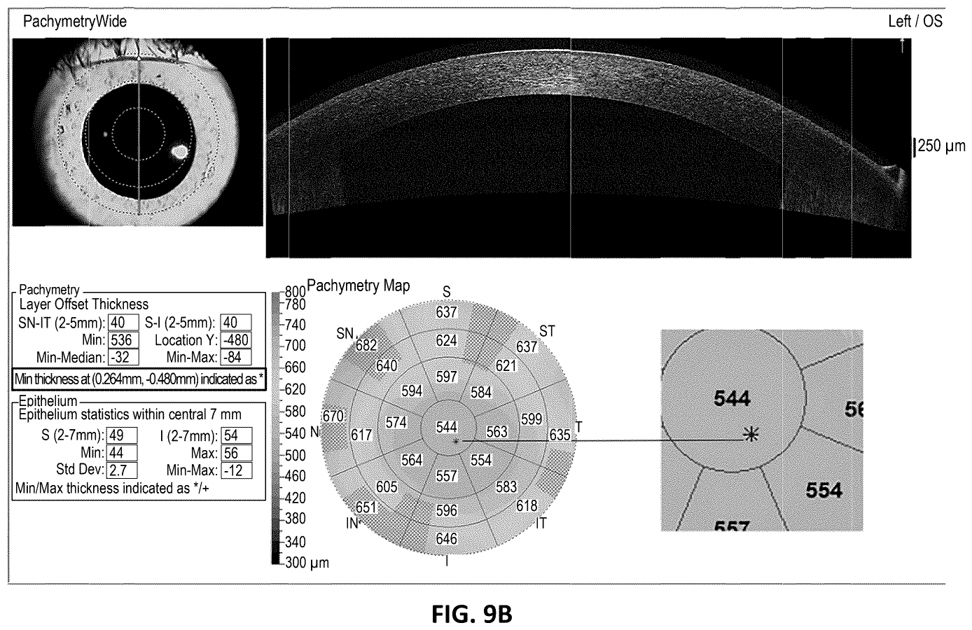

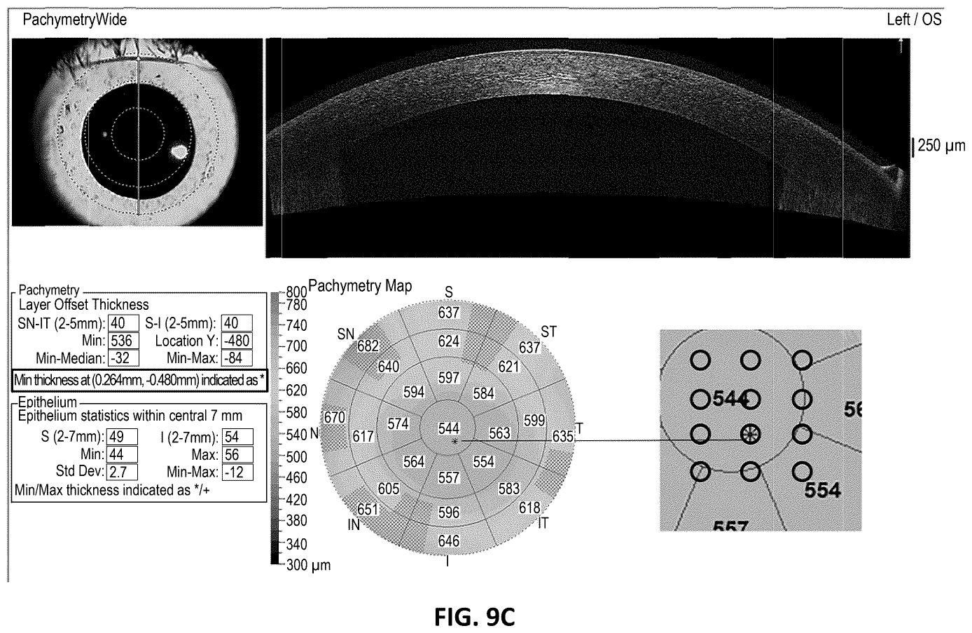

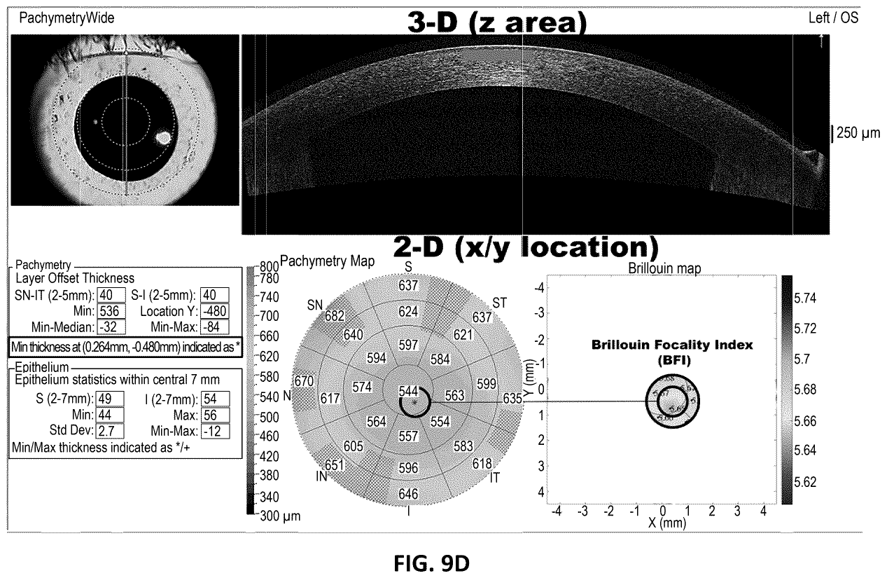

Thus, the location of MTB A-MIN co-localizes with the TCP for the vast majority of normal controls and SKC eyes. As such, this data can be used in performing focal guided evaluation for SKC screening, for example, by measuring Brillouin shifts in a localized region around the TCP that maximally differentiates normal corneas from those with SKC. A Brillouin focality index (BFI) can be derived utilizing data from this focal region, for example, based on a Brillouin shifts measured at points within 2-mm of the TCP. For example, a system with integrated OCT can obtain a regional corneal pachymetry map of a patient's cornea (5-mm to 8-mm), as shown in FIG. 9A. The TCP (star) can be automatically determined by software analysis, as shown in FIG. 9B. An automated positioning system (e.g., motorized translational stage) can align the patient's eye with interrogating light from the integrated Brillouin modality of the system. Brillouin shift measurements can then be obtained for the eye in a formatted grid pattern, as shown in FIG. 9C. For example, Brillouin measurements can be obtained at the TCP and for lateral positions within 2-mm of the TCP. As shown in FIG. 9D, the BFI can then be calculated using the Brillouin measurements, for example, post-processing combined data including a minimum Brillouin shift measured for a point within 1-mm of the TCP, Brillouin shifts measured for anterior stromal points to a depth of 200-μm of the cornea (or 40% of a thickness of the cornea), a maximum Brillouin shift, and/or a plateau length calculated based on the measured Brillouin shifts.

### CONCLUSION

Although the discussion above mentions “imaging,” the production of an actual image is not strictly necessary. Indeed, the mentions of “imaging” are intended to include the acquisition of data via the Brillouin and/or other imaging modalities where an image may not be produced. For example, the Brillouin spectrometer may produce graphical results of the Brillouin scattering or produce values (or a graphical display of values) corresponding to the measured physical properties of the sample. Similarly, the imaging modalities may produce data or other information used (e.g., in the processing of the Brillouin data, or otherwise) without an actual image being produced. Accordingly, the use of the term “imaging” herein is intended to include such scenarios and should not be understood as limiting.

The references to “light” used herein are not intended to define a specific wavelength for operation of the disclosed optical systems. Rather, the term “light” is intended to include any wavelength in the electromagnetic spectrum at which microscopes and/or Brillouin spectroscopy systems typically operate. Such wavelengths can include but are not limited to 350-1000 nm, inclusive.

Although particular optical components and configuration have been illustrated in the figures and discussed in detail herein, embodiments of the disclosed subject matter are not limited thereto. Indeed, one of ordinary skill in the art will readily appreciate that different optical components or configurations can be selected and/or optical components added to provide the same effect. In practical implementations, embodiments may include additional optical components, fewer optical components, or other variations beyond those illustrated, for example, additional reflecting elements to manipulate the beam path to fit a particular microscope geometry. Accordingly, embodiments of the disclosed subject matter are not limited to the particular optical configurations specifically illustrated and described herein.

Any of the features illustrated or described herein, for example, with respect to FIGS. 1A-9D, can be combined with any other feature illustrated or described herein, for example, with respect to FIGS. 1A-9D, to provide materials, systems, devices, structures, methods, aspects, or embodiments not otherwise illustrated or specifically described herein. All features described herein are independent of one another and, except where structurally impossible, can be used in combination with any other feature described herein. In view of the many possible aspects to which the principles of the disclosed technology may be applied, it should be recognized that the illustrated features are only examples and should not be taken as limiting the scope of the disclosed technology. Rather, the scope is defined by the following claims. Applicants therefore claim all that comes within the scope and spirit of these claims.

1. A system comprising:
a first imaging device configured to optically interrogate an eye of a patient;
a Brillouin spectroscopy device; and
a controller operatively coupled to the first imaging device and the Brillouin spectroscopy device so as to communicate therewith and control operational aspects thereof, the controller comprising one or more processors and one or more computer readable storage media storing computer readable instructions that, when executed by the one or more processors, cause the system to:
(a) measure one or more corneal geometry characteristics of the eye via the first imaging device;
(b) define a focal test region for the eye based at least in part on the measured one or more corneal geometry characteristics;
(c) obtain a plurality of Brillouin spectroscopy measurements of the eye for different measurement points via the Brillouin spectroscopy device;
(d) calculate a Brillouin focality index (BFI) based at least in part on one or more of the plurality of Brillouin spectroscopy measurements obtained for measurement points in the focal test region; and
(e) classify the eye as exhibiting subclinical keratoconus based at least in part on the calculated BFI.

2. The system of claim 1, wherein the controller is configured such that, in performing (c), the plurality of Brillouin spectroscopy measurements are obtained only for measurement points within the focal test region.

3. The system of claim 1, wherein the Brillouin spectroscopy device comprises a light source and a filter disposed in an optical path between the light source and the eye; and
wherein:
(i) the light source emits light having a bandwidth of at least 1-GHz;
(ii) the filter has a finesse of at least 100 for the light with respect to a predetermined wavelength;
(iii) the predetermined wavelength is about 780-nm;
(iv) the filter provides an extinction of at least 60-dB with respect to the predetermined wavelength;
(v) a combination of the light source and the filter is such that a bandwidth of the light directed to the eye is about 100-MHz; or
(vi) any combination of (i), (ii), (iii), (iv), and (v).

4. The system of claim 3, wherein the filter is a fiber-based Fabry-Perot filter.

5. The system of claim 1, wherein:
the measured one or more corneal geometry characteristics of the eye comprise corneal thickness; and
the focal test region is defined such that each of the different measurement points is laterally within 2 mm of a point in the eye having a minimum corneal thickness.

6. The system of claim 5, wherein the first imaging device is an optical coherence tomography (OCT) system configured to provide a 3-D reconstruction of corneal structure.

7. The system of claim 1, wherein:
the measured one or more corneal geometry characteristics of the eye comprise corneal curvature; and
the focal test region is defined such that each of the different measurement points is laterally within 2 mm of a point in the eye having a maximum corneal curvature.

8. The system of claim 7, wherein the first imaging device is a Placido ring imaging system.

9. The system of claim 1, further comprising:
a second imaging device configured to optically interrogate the eye,
wherein the first imaging device is configured to measure one of corneal thickness and corneal curvature, and the second imaging device is configured to measure the other of corneal thickness and corneal curvature.

10. The system of claim 1, further comprising a third imaging device configured to measure displacement of the eye in a lateral direction.

11. A method comprising:
optically interrogating an eye of a patient so as to measure one or more corneal geometry characteristics;
defining a focal test region for the eye based at least in part on the measured one or more corneal geometry characteristics;
obtaining a plurality of Brillouin spectroscopy measurements of the eye for different measurement points;
calculating a Brillouin focality index (BFI) based at least in part on one or more of the plurality of Brillouin spectroscopy measurements obtained for measurement points in the focal test region; and
classifying the eye as exhibiting subclinical keratoconus based at least in part on the calculated BFI.

12. The method of claim 11, wherein the measured one or more corneal geometry characteristics comprise corneal thickness, and the focal test region is defined such that each of the different measurement points is laterally within a predetermined distance from of a target point in the eye having a minimum corneal thickness.

13. The method of claim 12, wherein the predetermined distance from the target point is 2-mm or less.

14. The method of claim 12, wherein the obtaining the plurality of Brillouin spectroscopy measurements comprises obtaining Brillouin spectroscopy measurements at multiple depths from a front of the cornea for each lateral measurement position.

15. The method of claim 14, wherein:
the Brillouin spectroscopy measurements are obtained at multiple depths less than or equal 200-μm from the front of the cornea; or
the Brillouin spectroscopy measurements are obtained at multiple depths less than or equal to 40% of the measured corneal thickness at the respective lateral measurement position.

16. The method of claim 14, further comprising:
determining a plateau length for each lateral measurement position based on the corresponding Brillouin spectroscopy measurements at multiple depths.

17. The method of claim 16, wherein the BFI is calculated based on:
(i) a minimum Brillouin spectroscopy measurement obtained within the focal test region and for a measurement point that is laterally less than or equal to 1-mm from the target point,
(ii) a maximum Brillouin spectroscopy measurement obtained within the focal test region;
(iii) at least one of the determined plateau lengths; or
(iv) any combination of (i), (ii), and (iii).

18. The method of claim 14, wherein a time to obtain the plurality of Brillouin spectroscopy measurements for at least fifty different combinations of lateral measurement positions and depths is less than or equal to 1-minute.

19. The method of claim 11, wherein the measured one or more corneal geometry characteristics comprise corneal curvature, and the focal test region is defined such that each of the different measurement points is laterally within a predetermined distance from of a target point in the eye having a maximum corneal curvature.

20. The method of claim 11, wherein the obtaining is such that Brillouin spectroscopy measurements are obtained only for measurements points in the focal test region, and an acquisition time to obtain all of the Brillouin spectrometry measurements for the measurement points in the focal test region is less than 5-minutes.
